(19) United States
(12) Patent Application Publication
(10) Pub. No.: US 2023/0044505 A1
(43) Pub. Date: Feb. 9, 2023

(54) METHOD AND APPARATUS FOR BREATH-BASED BIOMARKER DETECTION AND ANALYSIS

(71) Applicant: VIRTUAL SENSE GLOBAL TECHNOLOGIES PRIVATE LIMITED, Mumbai (IN)

(72) Inventors: SANJAY PHADKE, MUMBAI (IN); GIRISH ARABALE, MUMBAI (IN)

(21) Appl. No.: 17/791,039

(22) PCT Filed: Jan. 6, 2021
(86) PCT No.: PCT/IN2021/050015

§ 371 Date: Jul. 6, 2022

(30) Foreign Application Priority Data
Jan. 6, 2020 (IN) 202021000591

(51) Int. Cl. A61B 5/08; G01N 27/12; A61B 5/097

(52) U.S. Cl. CPC A61B 5/082; G01N 27/125; G01N 27/128; A61B 5/097; A61B 2562/166; A61B 2562/046

(57) ABSTRACT

The present invention provides a device for non-invasive monitoring and/or detection of diabetes in a subject based on detection of volatile organic compounds (VOCs) in the exhaled breath of a subject. The device comprises a functionalized carbon nanotube-based array sensor which can reversibly bind VOCs, which alters the electrical conductivity of the sensor array, which can be interpreted to monitor and/or diagnose diabetes.

### FIELD OF INVENTION

The present invention relates to the field of diagnostic devices. In particular, the present invention relates to a device for non-invasive monitoring and detection of diabetes based on Volatile Organic Compounds (VOCs) in the breath of a subject.

### BACKGROUND OF THE PRESENT INVENTION

Diabetes is a chronic medical condition caused due to high blood sugar. In diabetes, the body does not produce sufficient insulin (type 1) or becomes resistant to insulin (type 2). Without timely diagnosis and treatment, diabetes can lead to blindness, kidney failure, and nerve damage. Diabetes is also an important factor in strokes and coronary heart diseases.

Timely detection and proper management can lead to improvement in lifestyle and diabetes can be managed without any significant loss of quality of life. However, early diagnosis is essential. In cases where clinical symptoms may not be present, if there is any underlying case of diabetes, detection can help with management of the disease.

An equally important aspect of management is monitoring of diabetes. Ease in monitoring can help in better management and change in medication or dosage if required.

While there are many blood-based detection and monitoring methods, they all are invasive and require specialized lab equipment and reagents, which not be available everywhere and are not portable, or at the very least, require a blood sample.

It is generally known in the art that the breath of a person can comprise various volatile organic compounds which may be indicative of the health status of a person. For example, an article by Szulejko et al., Evidence for Cancer Biomarkers in Exhaled Breath, IEEE Sensors Journal, Vol 10, No. 1 January 2010 is a review of presence of biomarkers in breath of cancer patients. Dogs are known to be able to smell odor signatures of cancer patients.

However, there is a lack of any device or system which can reliably detect diseases, such as diabetes based on the breath of a person and provide a diagnosis with a high reliability. The present invention provides such a non-invasive device which can be used to diagnose diabetes based on the breath profile of a subject.

### SUMMARY OF THE INVENTION

In an aspect of the present invention, there is provided a non-invasive device for monitoring and/or detection of diabetes in the breath of a subject, comprising: (a) a carbon nanotubes (CNTs) based sensor array; and (b) a printed circuit board, wherein the CNTs are functionalized with at least one of COOH, NH₃, and OH, wherein the CNTs reversibly bind at least one VOC present in the breath of the subject, and wherein the CNTs density per unit area of substrate in the array is in the range of 10-30 μg/mm², preferably in the range of 10-20 μg/mm².

In another aspect of the present invention, there is provided a method of monitoring and/or detecting diabetes comprising: (a) contacting the breath of a subject with a sensor array of a device comprising: (1) a carbon nanotubes (CNTs) based sensor array; and (2) a printed circuit board; (b) detecting the change in electrical conductance and/or resistance of the sensor array due to reversible binding of at least one VOC in the breath of the subject; (c) preparing at least one data point of the change in electrical conductance and/or resistance; (d) transmitting the at least one data point to an external server via a wired or wireless media; and (e) receiving by a mobile application processed result indicative presence or absence of diabetes; or status of pre-existing diabetes condition.

In yet another aspect of the present invention, there is provided a non-invasive comprising: (a) a carbon nanotube (CNTs) based sensor array; and (b) a printed circuit board for use in detecting and measuring at least a Volatile Organic Compound (VOC) in the breath of a subject for monitoring and/or diagnosis of diabetes.

This summary is not intended to identify essential features of the claimed subject matter nor is it intended for use in determining or limiting the scope of the claimed subject matter.

### BRIEF DESCRIPTION OF THE ACCOMPANYING DRAWINGS

FIG. 1: depicts the differential response of functionalized MWCNT-COOH to acetone and isoprene in accordance with an embodiment of the present invention.

FIG. 2: depicts the baseline corrected differential response of functionalized MWCNT-COOH to different concentrations of acetone, in accordance with an embodiment of the present invention.

FIG. 3: depicts the response of functionalized MWCNT-NH₃ to isoprene at concentrations varying from 0.05 PPM-1 PPM, in accordance with an embodiment of the present invention.

FIG. 4: depicts the response of functionalized MWCNT-OH to isoprene at concentrations varying from 0.2 PPM-1 PPM, in accordance with an embodiment of the present invention.

FIG. 5: depicts the response of functionalized MWCNT-OH to acetone at concentrations varying from 0.5 PPM-2 PPM, in accordance with an embodiment of the present invention.

FIG. 6: depicts the structural features of the non-invasive device of the present invention.

FIG. 7: depicts the schematic of preparing the CNTs based sensor array comprised in the device of the present invention.

### DETAILED DESCRIPTION OF THE INVENTION

Those skilled in the art will be aware that the invention described herein is subject to variations and modifications other than those specifically described. It is to be understood that the invention described herein includes all such variations and modifications. The invention also includes all such steps, features, compositions, and methods referred to or indicated in this specification, individually or collectively, and any and all combinations of any two or more of said steps or features.

For convenience, before further description of the present invention, certain terms employed in the specification, examples are collected here. These definitions should be read in light of the reminder of the disclosure and understood as by a person of skill in the art. Unless defined otherwise, all technical and scientific terms used herein have the same meaning as commonly understood by a person of ordinary skill in the art. The terms used throughout this specification are defined as follows, unless otherwise limited in specific instances.

The present disclosure is not to be limited in scope by the specific embodiments described herein, which are intended for the purposes of exemplification only.

The present invention provides a non-invasive device for monitoring and/or detection of diabetes in the breath of a subject, comprising: (a) a carbon nanotubes (CNTs) based sensor array; and (b) a printed circuit board, wherein the CNTs are functionalized with at least one of COOH, NH₃, and OH, wherein the CNTs reversibly bind at least one VOC present in the breath of the subject, and wherein the CNTs density per unit area of substrate in the array is in the range of 10-30 μg/mm², preferably in the range of 10-20 μg/mm².

In an embodiment, the device of the present invention is capable of detecting/diagnosing diabetes in a subject. In another embodiment, the device of the present invention is capable of monitoring status of pre-existing diabetes in a subject. The device is capable of measuring changes/modulations in VOCs levels in the breath of a subject with pre-existing diabetes condition so as to monitor diabetic status.

The device further comprises at least one of a humidity and temperature sensor; a flow sensor; a VOC enrichment module; and a moisture removal module. In an embodiment, the device comprises a humidity and temperature sensor, a flow sensor, and a VOC enrichment module. In an embodiment, a single module functions as moisture removal module and a VOC enrichment module. In an embodiment, the moisture removal module and a VOC enrichment module are enabled by way of silica gel, which removes moisture from the exhaled breath of a subject, resulting in enrichment of VOCs fraction in the exhaled breath.

The CNTs based sensor array is based on CNTs (single walled and/or multi walled carbon nanotubes) dispersed in a polymer which can be conducting or non-conducting solvent (conducting polymers can be chitosan, PEDOT, polyaniline and the like. Non-conducting polymers can be polyvinyl alcohol, polyalcohol and the like). The CNTs dispersed in the polymer are drop cast or spray using spray-gun on a substrate and dried to obtain the CNTs based sensor array. In an embodiment, the substrate can be acid free paper, Whatman filter paper, cellulose based, polymer based, metal based, and combinations thereof. In a preferred embodiment, the substrate is Whatman free paper and acid free paper based. In a particular embodiment, the substrate is paper. In a preferable embodiment, the CNTs are multi walled. In an embodiment, the concentration of functionalized CNTs per unit area of substrate is in the range of 10-30 μg/mm². In a preferred embodiment, the concentration of functionalized CNTs per unit area of substrate is in the range of 10-20 μg/mm².

The CNT based sensor array works on the principle of detection of change in electrical properties such as resistance and/or conductance upon binding of a chemical compound (such as VOC) as a function of time. The time component is divided into initiation time, sensing time, readout time, and processing time. The concentration of chemical compounds (such as VOC) may also induce a differential change in the electrical properties of the array.

In an embodiment, the CNTs are functionalized with at least one of COOH, NH₃, and OH functional groups. Functionalization of CNTs can be carried out in any known process in the art.

The CNTs of the sensor array are capable of reversibly binding VOCs, such as at least one of acetone and isoprene. In an embodiment, a first subset of functionalized CNTs binds acetone. In another embodiment, a second subset of functionalized CNTs binds isoprene. In an embodiment, a single set of functionalized CNTs binds acetone and isoprene. In an embodiment, binding of acetone or isoprene induces a detectable and measurable change in the electrical properties of the array. In an embodiment, the measurable change in the electrical properties of the array upon binding of acetone is distinct from the measurable change in the electrical properties of the array upon binding of isoprene. In an embodiment, the sensor array of the present invention comprises a first and second subset of functionalized CNTs capable of binding acetone and isoprene, respectively. In an embodiment, the sensor array of the present invention comprises a single set of functionalized CNTs capable of binding acetone and isoprene.

Binding of VOCs to the CNTs on the sensor substate results in a change of electrical conductance of the array. The change in electrical conductance of the array is detectable and measurable. The array can measure the differential binding of acetone or isoprene. The array can also measure the relative concentration of acetone or isoprene bound to the array.

In an embodiment, the sensitivity of detection of binding of VOCs to the CNTs based sensor array is 1 PPB (parts per billion). In an embodiment, detection of acetone at a concentration of at least 2 PPM (parts per million) is indicative of diabetes in the subject. In another embodiment, detection of isoprene at a concentration of at least 1 PPM is indicative of diabetes in the subject.

In another embodiment, detection of acetone and isoprene at a concentration of at least 2 PPM and 1 PPM respectively is indicative of diabetes in the subject.

It is understood to a person skilled in the art that the acetone and/or isoprene concentration levels which are indicative of diabetes are based on standard diet and health conditions. There may be specific health situations or lifestyle situations (such as, but not limited to keto diet for instance) which may result in increase in VOCs in the breath that may give a false positive indication.

In an embodiment, the detection accuracy of the device of the present invention is in the range of 95-98%.

The sensor array is mounted in a cassette, which is made of a printed circuit board (PCB) sandwiched with the sensor array (as a strip) in the middle. In an exemplary embodiment, the lower PCB is extended to have a USB A male interface which plugs into a USB A female interface in the apparatus. The read out is executed using a four prong/pin of the USB A male interface. The USB A pins are connected to the sensor strip via etched tracks on the lower board.

The electronics/readout board is a microcontroller-based system with a USB A female interface fixed (in different configurations such as right angle, in parallel) in a manner such that the cassette when inserted into the USB port is exposed to the breathing chamber where the breath of a subject is collected. The electronics are designed to readout the resistance/conductance of the sensor strip as a function of time using a constant current source and a Wheatstone bridge) with an accuracy of about 0.0001 Ohm. In an alternative embodiment, the total impedance may be measured as a function of time. In another embodiment, the current may be measured as a function of time with constant voltage. The readout board is powered by a built-in battery, which in an embodiment can be rechargeable. The board can communicate with external devices via one or more known wired or wireless interfaces, such as Bluetooth, WiFi, Ethernet, and the like. The software module of the device of the present invention includes functionality to measure the change(s) in resistance in the CNTs array due to reversible binding of one or more particular VOCs present in the breath.

The firmware of the device of the present invention initiates the readout by sampling resistance in the CNTs based sensor array at a constant rate. In an embodiment, sampling can at 125 ms, 250 ms, 500 ms period and the like. In an embodiment, sampling rate can be varied. Data is collected for a pre-defined rate of 1 s, 2 s, 5 s, 10 s and the like. In an embodiment, the data collection rate can be varied. In an embodiment, each data collection point is tagged in a sequential manner. In an embodiment, the tag can be a time stamp. In another embodiment, the tag can be a counter.

In an embodiment, the data points may be communicated to an external server via a wired or wireless network known in the art, such as WiFi, Bluetooth, etc. In another embodiment, the data points may be transferred to an external storage media (such as compact disc, solid state drive, pen drive, and the like) prior to uploading on an external server. In an embodiment, the data points may be cached in the device.

The data points are processed by algorithmic comparison with reference data points. In an embodiment, the algorithm may be Al or ML enabled. Reference data points may be obtained under controlled laboratory conditions. In an embodiment, the comparison may include at least one of power law indices of the growth and decay curve of the response, integrated features like the area under the curve, saturation values, saturation times, etc.

The results, in an embodiment, may be communicated to a mobile application, results comprising identification of VOCs and concentration of VOCs. In the case of diabetes, for instance, detection of acetone at a concentration of at least 2 PPM and detection of isoprene at a concentration of at least 1 PPM would be indicative of diabetes in the subject. The mobile application can display the data in real-time. In an embodiment, the mobile application can also provide multiple results collected over multiple discrete time points to provide a historical perspective.

The device of the present invention is also capable of monitoring changes in VOCs in the breath of a subject with pre-existing diabetes.

The present invention also provides a method of monitoring and/or detecting diabetes, comprising: (a) contacting the breath of a subject with the sensor array of a device as substantially described herein; (b) detecting the change in electrical conductance and/or resistance of the sensor array due to reversible binding of at least one VOC in the breath of the subject; (c) preparing at least one data point of the change in electrical conductance and/or resistance; (d) transmitting the at least one data point to an external server via a wired or wireless media; and (e) receiving by a mobile application processed result indicative of presence or absence of diabetes; or status of pre-existing diabetes condition.

The present invention also provides a non-invasive device as substantially described herein for use in detecting and measuring at least a Volatile Organic Compound (VOC) in the breath of a subject for monitoring and/or diagnosis of diabetes.

### EXAMPLES

The invention will now be illustrated with working examples, which is intended to illustrate the working of the invention and not intended to be taken restrictively to imply any limitations to the scope of the present invention. Unless otherwise defined, all technical and scientific terms used herein have the same meaning as commonly understood to one of ordinary skill in the art to which this disclosure belongs.

In a non-limiting exemplary embodiment, —COOH functionalization protocol is provided below: (1) preparing 8M HNO₃ and 8M H₂SO₄ stock solution of 100 mL each; (2) weighing 300 mg multiwalled carbon nanotubes (MWCNT); (3) preparing 70 mL acid solution (35 mL each of prepared HNO₃ and H₂SO₄) and mixing MWCNT in acid solution while stirring for 20 mins at 60° C.; (4) sonicating for about 3 hours; and (5) filtering the slurry, PTFE 6, 6 membranes (0.45 micron) (Polytetrafluoroethylene), and wash liquid volume 100 mL.

In another non-limiting exemplary embodiment, —NH₃ functionalization protocol is provided below: (1) The carboxylic groups of the MWCNTs were activated by using a solution of 0.1 mol L⁻¹ acetate buffer (pH 4.8) containing 0.1 mol L⁻¹ EDC (N-ethyl-N-(3-dimethylaminopropyl) carbodiimide) and 0.2 mol L⁻¹ NHS (N-hydroxysuccinimide) [1:1]; (2) At the same time, amino groups of the EDA were submitted to acid treatment in H₂SO₄ solution (0.1 mol L⁻¹) for 2 h under stirring conditions at 60° C., changing their protonation state; (3) subsequently, 1.0 mg activated MWCNTs was dispersed in 1.0 μL EDA treated and it was stirred for 2.5 hours at room temperature; (4) The sample was centrifuged at 2000 rpm and washed five times with deionized water to remove acid residues; and (5) The resulting amino-MWCNTs were dried at 150° C. and then dispersed in water under sonication during 2 hours.

Functionalization of MWCNT for different functional groups observed using the Fourier Transform Infrared Spectroscopy (FTIR) was quantitatively estimated and found to be 9.26% (OH group), 32.80% (NH₃ group) and 39.96% for COOH group.

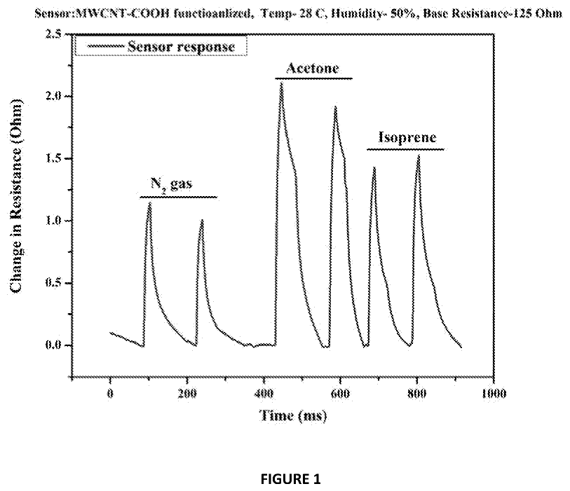

In an exemplary example, FIG. 1 shows the differential response of functionalized MWCNT-COOH to acetone and isoprene. It can be readily seen that MWCNT-COOH can differentially detect both acetone and isoprene in the breath of a subject.

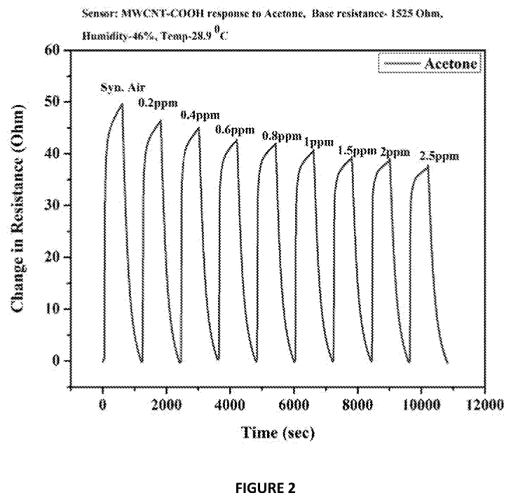

In an exemplary example, FIG. 2 shows the baseline corrected differential response of functionalized MWCNT-COOH to different concentrations of acetone. It can be appreciated that MWCNT-COOH can differentially detect acetone at varying concentrations.

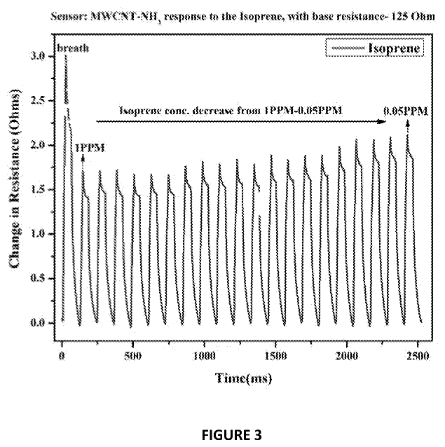

In an exemplary example, FIG. 3 shows the response of functionalized MWCNT-NH₃ to isoprene at concentrations varying from 0.05 PPM-1 PPM. It can be appreciated that MWCNT-NH₃ can differentially detect isoprene at varying concentrations.

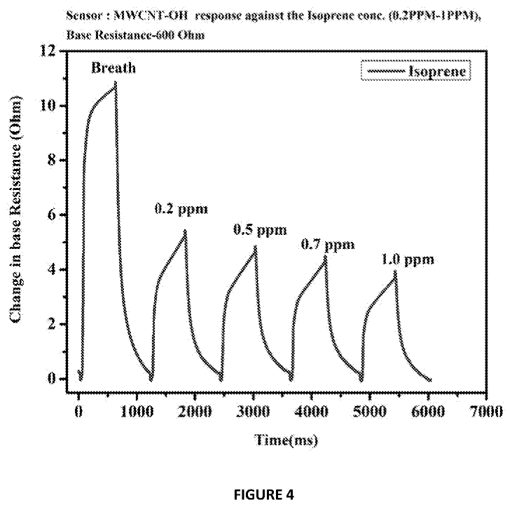

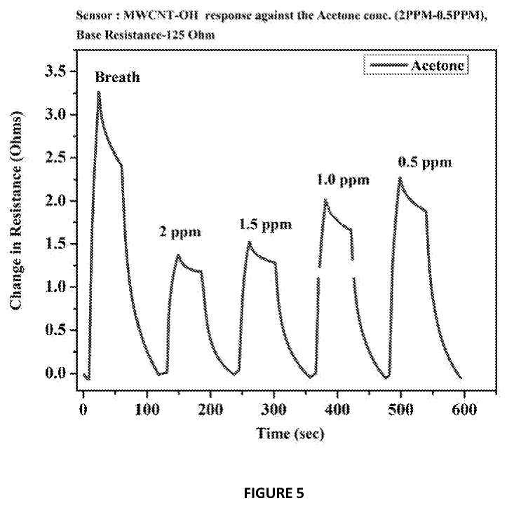

In another exemplary example, FIG. 4 shows the response of functionalized MWCNT-OH to isoprene at concentrations varying from 0.2 PPM-1 PPM. FIG. 5 shows the response of functionalized MWCNT-OH to acetone at concentrations varying from 0.5 PPM-2 PPM. It can be appreciated that MWCNT-OH can differentially detect isoprene and acetone at varying concentrations.

In accordance with the objectives of the present invention, Table 1 below summarizes the detection abilities of a device of the present invention.

TABLE 1

| | | | No. of | |
|---|---|---|---|---|
| S. No. | Trial date | Venue | volunteers | Remarks |
| 1 | 18 Sep. 2019 | Clinic 1 | 4 | Type-1 |
| 2 | 18 Oct. 2019 | Clinic 2 | 37 | Healthy/diabetic/thyroid |
| 3 | 19 Oct. 2019 | Clinic 3 | 5 | Diabetic |
| 4 | 24 Oct. 2019 | Clinic 2 | 24 | Diabetic |
| 5 | 25 Oct. 2019 | Clinic 2 | 11 | Diabetic |
| 6 | 26 Oct. 2019 | Clinic 2 | 3 | Diabetic |
| 7 | 1 Nov. 2019 | Clinic 4 | 17 | Diabetic |
| 8 | 2 Nov. 2019- | Clinic 4 | 6 | Diabetic |
| | 7 Nov. 2019 | | | |
| 9 | 24 Nov. 2019 | Clinic 5 | 77 | Diabetic |
| 10 | 25 Nov. 2019 | Clinic 5 | 5 | Diabetic |
| 11 | 27 Nov. 2019 | Clinic 2 | 14 | Diabetic |
| 12 | 1 Dec. 2019 | Clinic 5 | 38 | Type-1 |
| 13 | 3 Dec. 2019 | Clinic 5 | 8 | Diabetic |
| 14 | 11 Dec. 2019 | Clinic 2 | 21 | Diabetic |
| 15 | 26 Dec. 2019 | Clinic 6 | 48 | Healthy/diabetic |
| 16 | 28 Dec. 2019- | At our lab | 19 | Healthy |
| | 31 Dec. 2019 | | | |

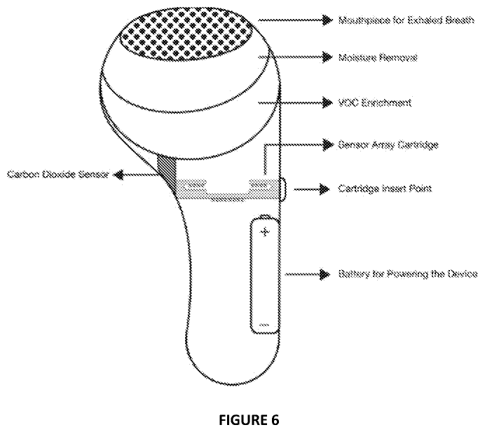

In an exemplary example, FIG. 6 depicts the structural features of the non-invasive device of the present invention. As seen in FIG. 6, the device comprises a mouthpiece into which exhaled breath of a subject can be collected. Subsequently, there is a moisture removal and VOC enrichment module. Moisture from the breath is removed by aid of silica gel, which also consequently enriches the VOC fraction in the breath. The VOC enriched sample is subsequently exposed to the CNTs based sensor array, which is removable. The device also comprises a power source connected to the CNTs based sensor array. The device further comprises a PCB/firmware module (not shown), which is capable of directing the detection and measurement of any changes in the electrical conductance of the sensor array upon binding of VOCs to the array. The changes are recorded and transmitted (via wired or wireless media) to a server for further processing (not shown). The results (concentration of VOCs indicative of diabetes (acetone and/or isoprene)) are communicated to a mobile application for the user. The device also comprises a carbon dioxide sensor. The device also comprises VOC egress area (not shown). The device can be reset (removal of bound VOCs) by passing air through the device for a specified period of time.

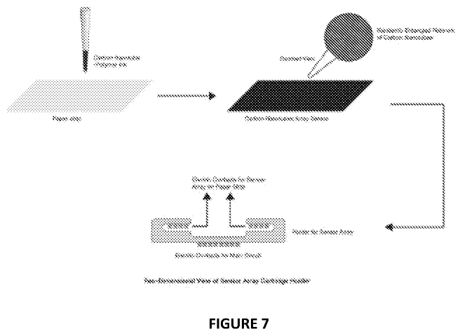

In an exemplary example, FIG. 7 depicts the schematic of preparing the CNTs based sensor array. As described in detail in the description and prior examples, a homogenous mix of multiwalled carbon nanotubes in a polymer ink (chitosan) is deposited on a paper substate at a concentration of 10-30 μg/mm², preferably 10-20 μg/mm² whereby the carbon nanotubes form a random entangled network on the substrate. The array is positioned in the cassette as shown in FIG. 7.

### ADVANTAGES OF THE PRESENT INVENTION

The present invention provides a non-invasive device for accurate, sensitive, and reproducible detection of diabetes based on differential detection of VOCs in the breath of a subject. The device does not use any consumables and therefore has a low overall cost and can be used in various settings where lab equipment may not be available. The results generated by the device can be electronically coded and processed easily. The reversal time is also very fast compared to traditional methods.

We claim:

1. A non-invasive device for monitoring and/or detection of diabetes in the breath of a subject, comprising:
a. a carbon nanotube (CNTs) based sensor array; and
b. a printed circuit board,
wherein the CNT are functionalized with at least one of COOH, NH₃, and OH, wherein the CNT reversibly bind at least one Volatile Organic Compound (VOC) present in the breath of the subject, and wherein the CNT density per unit area of substrate in the array is in the range of 10-30 μg/mm², preferably in the range of 10-20 μg/mm².

2. The device as claimed in claim 1, further comprising at least one of a humidity and temperature sensor; a flow sensor; a VOC enrichment module; and a moisture removal module.

3. The device as claimed in claim 1 wherein said VOC is at least one of acetone, and isoprene.

4. The device as claimed in claim 1, wherein binding of at least one VOC to the CNTs induces a detectable and measurable change in the electrical conductance of the array.

5. The device as claimed in claim 1, wherein detection of acetone binding equal to or more than 2 PPM is indicative of diabetes condition, and wherein detection of isoprene binding equal to or more than 1 PPM is indicative of diabetes condition.

6. The device as claimed in claim 1, wherein the detection accuracy is in the range of 95-98%.

7. The device as claimed in claim 1 further comprising a software module capable of connecting with a network over a wired or wireless media.

8. A method of monitoring and/or detecting diabetes comprising:
a. contacting the breath of a subject with the sensor array of a device as claimed in claim 1;
b. detecting the change in electrical conductance and/or resistance of the sensor array due to reversible binding of at least one VOC in the breath of the subject;
c. preparing at least one data point of the change in electrical conductance and/or resistance;
d. transmitting the at least one data point to an external server via a wired or wireless media; and
e. receiving by a mobile application processed result indicative of presence or absence of diabetes; or status of pre-existing diabetes condition.

9. The method as claimed in claim 8, wherein the subject blows into the device for contacting of the breath of the subject with the sensor array.

10. The method as claimed in claim 8, wherein the detection accuracy is in the range of 95-98%.

11. A non-invasive device as claimed in claim 1 for use in detecting and measuring at least a Volatile Organic Compound (VOC) in the breath of a subject for monitoring and/or diagnosis of diabetes.
